(19) United States
(12) Patent Application Publication
(10) Pub. No.: US 2008/0299103 A1
(43) Pub. Date: Dec. 4, 2008

(54) Well Preserved Aqueous Organic Compositions

(76) Inventors: Liliana George, Centerport, NY (US); Steve Schnittger, Huntington Station, NY (US); Kathy Kretzschmar, Smithtown, NY (US); Charles Craig Tadlock, Islip Terrace, NY (US); Monica Apostol, Coram, NY (US)

Correspondence Address: Peter Giancana, Suite 345 South, 155 Pinelawn Road, Melville, NY, 11747, US

(21) Appl. No.: 11/756,751

(22) Filed: Jun. 1, 2007

(51) Int. Cl. A61K 36/752; A61K 33/18; A61K 36/02; A61K 36/28; A61K 36/42; A61K 36/45; A61K 36/47; A61K 36/76; A61K 36/899; A61Q 19/00; A61K 38/44; A61K 36/889; A61K 36/53; A61K 36/54; A61K 36/61; A61K 36/63; A61K 36/73; A61K 36/736

(52) U.S. Cl. 424/94.4; 424/195.17; 424/669; 424/727; 424/731; 424/732; 424/735; 424/736; 424/739; 424/745; 424/750; 424/758; 424/764; 424/765; 424/776; 424/777; 424/778

(57) ABSTRACT

Well-preserved aqueous topical compositions that satisfy one or more widely recognized organic certification standards. The compositions comprise a preservative system comprising an in situ hypoiodite generator; and a plant extract blend; optionally, an essential oil blend. Such compositions do not require synthetic preservatives, especially parabens. Suitable aqueous compositions may contain an oil phase, such as oil-in-water or water-in-oil emulsions.

### FIELD OF THE INVENTION

The present invention is in the field of topical personal care products and specifically concerns preservative systems suitable for aqueous products that may be certified organic.

### BACKGROUND

Consumer demand for organic products is well established in many parts of the world. While we may generally think of food products in relation to organic certification, there is also a growing market for organic non-food items, including cosmetic and dermatologic compositions. The following was reported in Cosmetics & Toiletries magazine (Oct. 2, 2006):
Organic personal care products were reported to achieve US$282 million in sales in 2005, according to the Organic Trade Association's 2006 Manufacturer Survey. According to the survey, non-food organic product sales totaled US$744 million, which was up 28% from 2004. The survey also reported that 61% of respondents for food and non-food categories displayed the USDA organic seal on their products, many of them responding that the seal helped to sell their products.

The definitions of “organic product”, “organic ingredient”, “certified organic” and the like, have been evolving for some time, and international standards are only slowing converging. Government authorities have developed organic standards for cosmetics labeling to varying degrees. In the United States for example, a task force headed by the National Standards Foundation is due to draft standards for organic personal care products, by Summer 2007. It is expected that the proposed organic personal care standards will be similar to the organic foods standards promulgated by the US Department of Agriculture (USDA) through its National Organic Program (NOP). Currently, personal care products certified under USDA regulations may bear one of the approved logos, but there are no regulations in place to prevent non-certified products from labeling their products “organic”. The laws and regulations of the National Organic Program are promulgated under 7 U.S.C. 94.6501-94.6523 and 7 C.F.R. 205, herein incorporated by reference, in their entirety.

In Japan, organic products are defined under the Organic Japanese Agricultural Standard (JAS). In India, it's the NPOP Regulation (National Programme for Organic Production). In some parts of the world non-governmental organizations are proliferating standards for organic products and offering certification services. Organizations like Ecocert International offer organic cosmetic certification based on standards developed by Ecocert. Ecocert also verifies organic product standards against regulations in Europe, Japan and the United States. Other European-based organic cosmetic certifying organizations include the Soil Association Standards for Health and Beautycare Products, in Great Britain, and AIAB (Associazione Italiana per l'Agricoltura Biologica) Regulation, in Italy. These and other organizations are actively seeking harmonization, with the aim of proposing a uniform European regulation for organic cosmetic certification. In April, 2007, The Organic Farmers & Growers, an organic food inspector and licensor in Great Britain, announced its own standard for organic certification of cosmetics and personal care products. The standard is reportedly based on EU Organic Regulation (EC2092/91) and the Nordic Ecolabelling Standards. In Europe, Ecocert cosmetic certification is, perhaps, the most sought after certification. Although the Ecocert certification for cosmetics is voluntary, Ecocert's Cosmetics Department has, according to their website, certified more than 130 companies and more than 18,000 cosmetic products worldwide. Thus, a demand is growing among producers, handlers, processors and retailers, for organic certification services based on regionally and globally recognized standards.

At the time of filing this application, the USDA has yet to promulgate rules specific to the cosmetic industry. However, officially, the cosmetics industry is permitted to apply for NOP certification by adhering to the rules as they exist for the food industry. It should be borne in mind that the NOP standards were written for agricultural production and address the methods, practices and substances used in producing and handling crops, livestock, and processed agricultural products. The NOP requirements apply to the way the product is created and not necessarily to measurable properties of the product itself.

It is important to note that the terms “organic” and “natural” do not have the same meaning. In the present context, natural refers to not being synthetically or artificially produced. “Organic” also refers to the method of production, as well as methods of handling, storing, processing, packaging, shipping, etc. Organically produced food cannot be produced using excluded methods, and the same holds for cosmetics, even when all of the ingredients are natural. Thus, a NOP certified cosmetic product not only has the requisite level of “natural” ingredients, but the methods of production and handling of the ingredients and final product also conform to defined standards. This is a very stringent requirement for cosmetics and comparatively few cosmetics on the market, today, bear a NOP certification. According to “E-BEAUTY News” (no. 82), a beauty industry newsletter published by beauty-on-line.com, “In practice, these stringent requirements prevent from using the NOP Final Rule for the certification of most cosmetic products.”

Typically, NOP standards for organic certification include one or more lists of approved and disapproved materials, categorized by functions, like preservatives, sunscreens and colorants, to name a few. As a general rule, most natural (non-synthetic) substances are allowed in organic production, while most synthetic substances are prohibited. Specific exceptions to the general rule are found in The National List of Allowed Synthetic and Prohibited Non-Synthetic Substances (7 C.F.R. 205.600-205.606), herein incorporated by reference, in its entirety.

The National Organic Program sanctions the use of four levels of organic labeling. These are: “100 percent Organic”, which means, in part, that a product contains 100 percent certified organic ingredients, not counting added water and salt; “Organic”, which means that a product contains at least 95% certified organic ingredients, not counting added water and salt and the remaining ingredients (up to 5% percent non-organic) come from the National Organic Standards Board's list of allowable substances; “Made with Organic Ingredients”, which means that a product contains at least 70% certified organic ingredients, not counting added water and salt; and if a label merely claims that a product contains organic ingredients, the implication is that the product contains less than 70% certified organic ingredients.

At present, Ecocert organic certification for cosmetics provides two labels. A “Natural Cosmetic” comprises “5% minimum of Organic Certified ingredients on the total of ingredients, which represents 50% of vegetable ingredients”. A “Natural And Organic Cosmetic” comprises “10% minimum of Organic Certified ingredients on the total of ingredients, which represents 95% of vegetable ingredients”. For both labels, at least 95% of the ingredients must be of “natural origin”, which is to say that no more than 5% of synthetic ingredients are permitted. Like the NOP standard in the US, those synthetic ingredients must come from an approved list.

Thus, all important standards in use today, allow for some level of synthetic materials in the final product. The Ecocert list of approved synthetic ingredients comprises preservatives and processing aids. By allowing synthetic preservatives, the Ecocert Organic Cosmetic standard is acknowledging that preservatives of natural origin may not be sufficiently effective in cosmetic products. Thus, the ECOCERT standard for organic cosmetics is less stringent than the NOP standard, which may account for the greater number of cosmetic products bearing an ECOCERT organic label.

The invention of a preservative system of natural origin would be beneficial because a cosmetic containing a natural preservative system is potentially certifiable by NOP, as organic. Certainly, a preservative system that meets NOP certification standards removes a significant hurdle, perhaps the greatest hurdle, in marketing NOP-certified organic cosmetics. Furthermore, Ecocert and perhaps other certification standards, specify a maximum level of synthetic ingredients in a product. Those certification standards are more easily achieved if the preservative system is natural and does not count against the permitted level of synthetic ingredients. Thus, a natural preservative system would greatly improve the ability of cosmetic manufacturers to achieve the two most sought after organic certifications, NOP and Ecocert, as well as increase the types of products that may be implemented as all natural.

An acceptable cosmetic should be preserved against, or contain an antimicrobial agent effective against, at least the following groups of microorganisms: molds (such as, Aspergillus niger), yeasts (such as, Candida albicans), gram positive bacteria (such as Staphylococcus aureus), gram negative bacteria (such as Pseudomonas aeruginosa), and enteric bacteria (such as E-coli). Standards for preservation vary, but preservation testing is typically based on introducing a known level of microbial contamination into a product and then measuring the rate of kill over time. Details of preservation testing methods are promulgated by various organizations, including, for example, the US Pharmacopoeia and the CTFA. The US Pharmacopoeia and the CTFA employ a preservation standard of not more than 3 log reduction. Some manufacturers employ standards that are more rigorous than 3 log reduction. For example, it is not uncommon for antimicrobial efficacy of marketable products to be based on a 5 log reduction standard. 5 log reduction means that, within some defined period of time (seven days, for example), there is a 100,000 fold decrease in the number of bacteria, yeast and mold organisms present in the product. Thus, preservative systems that meet a 3 log reduction standard, in marketable cosmetic compositions, may be considered antimicrobially-effective, but a 5 log reduction standard is preferred by many producers and manufacturers. Furthermore, adequate preservation means that a product meets one or more preservation standards at various stages of development; i.e. in lab batches, in pilot scale up, in full scale production of marketable goods, in the hands of the consumer.

There are materials of natural origin that have preserving properties because they kill, prevent or otherwise inhibit microbial growth in situ. Various essential oils and plant extracts may fall into this category. However, at concentrations that are acceptable for topical products, the scope and duration of protection provided by these materials, is generally insufficient. Cosmetic and other topical products typically need to be preserved for weeks, months or even years against a broad spectrum of microbes. Some improvement in the situation is achieved by combining several essential oils and/or natural origin materials, but, in general, this has not led to an antimicrobial preservative system comparable to those of synthetic origin, that have become conventional in the cosmetic industry because of their broad applicability. Furthermore, essential oils and plant extracts introduce properties that may not be desirable in every product, like odor, irritation or allergic reaction.

U.S. Pat. No. 5,306,707 discloses perfume components of specific chemical structures that are antimicrobially effective in compositions comprising at least 25% water. A perfume component (not necessarily an essential oil) is “antimicrobially effective” if it requires at least three microbial inoculations to failure in a challenge test consisting of two microbes, Pseudomonas cepacia and Enterobacter cloacae. The product itself is considered preserved if the product also requires at least three microbial inoculations to failure in a challenge test. When read for all it discloses, the '707 reference discloses exactly one combination of perfume components that when used in shampoo or skin lotion, meets its own definition of what is antimicrobially effective. That combination is phenylacetic acid, cinnamic acid, phenylacetaldehyde, 2-methyl-2-hepten-6-one, phenylethyl formate, cis-3-hexenyl acetate, prenyl acetate, benzyl formate, cinnamic aldehyde. And furthermore, those nine perfume components were prepared in solution with at least 19 other compounds whose combined effect on microbes is unknown. The large number of perfume components needed to achieve some level of preservation, makes this system impractical for broad use in the plethora of cosmetic products that inhabit the marketplace. Furthermore, preservation was only demonstrated against Pseudomonas cepacia and Enterobacter cloacae. Effectiveness against molds (such as, Aspergillus niger), yeasts (such as, Candida albicans) and gram positive bacteria (such as Staphylococcus aureus), was not demonstrated. Thus, the '707 reference fails to disclose a well preserved aqueous, cosmetic or personal care composition.

U.S. Pat. No. 4,966,754 discloses a method of preserving a cosmetic composition from the microbial action of Aspergillus niger, Candida albicans, Staphylococcus aureus and Pseudomonas aeruginosa, comprising incorporating into a cosmetic composition a mixture of essential oils: Linalool (ex. Bois de Rose), Geraniol (ex. Palmarosa), Lemongrass 80% rectified, Bois de Rose, Cedarwood Oil, Marjoram Oil, Cinnamon Bark Oil, Cardamon Oil, Neroli Bigarde Petals Oil, Vanilla Resinoid, Coriander Oil, Oakmoss empuree, Armoise Oil, Menthol Crystals laevo, Rose absolute concrete (wax) and wherein the antimicrobial essential oil is incorporated into the cosmetic composition by first dissolving the antimicrobial essential oil in a polyoxyethylene sorbitan ester wherein the ratio by weight of the sorbitan ester to the antimicrobial essential oil is in the range of 2:1 to 6:1 and adding the resulting sorbitan ester and essential oil mixture to the cosmetic composition in an amount to provide the antimicrobial essential oil in the cosmetic composition of at least 2% by weight of the cosmetic composition. Here again, the essential oil mixture is specific and impractical for broad use. Furthermore, preservation was not demonstrated against enteric bacteria (such as E-coli). Thus, the '754 reference fails to disclose a well preserved aqueous, cosmetic or personal care composition.

Essential oils, in general, have very limited water solubility. Notwithstanding specific exceptions that may be found in the prior art, essential oils provide only limited protection for aqueous systems or aqueous phases of multiphase systems. Furthermore, to the extent that some essential oils (i.e. tea tree, citrus) and plant extracts provide preservative activity, they can also have adverse properties when applied to the skin in antimicrobial-effective quantities. Allergic reactions and generalized skin irritation are common concerns. Furthermore, natural preservatives themselves tend to deteriorate over time and lose efficacy, more so than synthetic preservatives. If that happens, the cosmetic composition would be subject to microbial attack and spoilage. Also of concern is the odor imparted by essential oils and the higher cost of natural origin ingredients compared to conventional synthetic preservatives. Because of its limitations, the use of essential oils and plant extracts for preservation has never achieved widespread use. Furthermore, essential oils are typically added to an oil phase, wherein the essential oils have their greatest preservation activity. If the oil phase is sequestered from the water phase, i.e. an oil-in-water emulsion, the essential oils may have little preservation effect in the water phase. Thus, water based and water containing cosmetic compositions commonly use alcohol as a preservative. In some certified organic products, in order to achieve adequate preservation, concentrations of alcohol in an aqueous phase may be as high as 15-20%. This level is too high for many types of personal care products. Such a high alcohol level may limit the types of products that a formulator can design or interfere with the aesthetic of the product. On the other hand, at lower levels of alcohol, sufficient preservation will probably not be achieved. Also, in NOP certified organic products (and perhaps other standards), the alcohol must be denatured in a manner proscribed by regulation. The cost and availability of certified organic alcohol may also be disadvantageous.

There remains a need for an effective preservative system (one that meets a 5 log reduction standard for molds, yeasts, gram positive bacteria, gram negative bacteria, and enteric bacteria), that has broad use in various types of cosmetics, particularly aqueous cosmetics that are certifiable organic.

Hypoiodite and hypothiocyanate are naturally occurring compounds known to have antimicrobial properties that make them effective preservation agents in aqueous preparations. One example of a system that generates hypoiodite and hypothiocyanate, in situ, is known as Biovert®, and is commercially available from Arch Personal Care. This system is described in U.S. Pat. No. 5,607,681 (herein incorporated by reference, in its entirety) and one example of its operation is as follows. A host system requiring preservation, such as an aqueous cosmetic preparation, is provided. Biovert is a two part system. The first is a substrate solution of glucose mixed with one or more salts of iodide and thiocyanate. Suitable iodide salts include potassium and sodium salts. Suitable thiocyanate salts include potassium, sodium, ammonium, ferric and cuprous salts. When introduced into the substrate solution, the iodide and thiocyanate ions are released from the salts. The substrate solution is incorporated into the aqueous cosmetic preparation thus infusing the preparation with iodide and thiocyanate ions. The second part of the Biovert® system is an enzyme solution comprising glucose oxidase and lactoperoxidase. In the presence of oxygen, which is available at the exposed surface of the preparation, glucose oxidase is broken down and hydrogen peroxide, H₂O₂, is released into the preparation. At this point, the original preparation is infused with iodide and thiocyanate ions, lactoperoxidase and hydrogen peroxide. In the presence of hydrogen peroxide (H₂O₂), lactoperoxidase is capable of oxidizing the iodide ion into hypoiodite (OI⁻) and the thiocyanate ion into hypothiocyanate (OSCN⁻). After being generated in situ, hypoiodite and hypothiocyanate go to work against microbes in the aqueous preparation. This system is reportedly effective against bacteria, yeast and mold.

This system has the feature that when the supply of oxygen is cut off, as when the container holding the preparation is sealed, the oxygen in the container is depleted. When the oxygen seal is broken, the preservative system immediately goes back to work. There are two advantages to this feature. Firstly, without oxygen, the cascade of reactions is halted so that the preservative system is not depleted. Secondly, because oxygen in or near the product is scavenged, the preparation is protected from oxidative damage. The use of the Biovert® type system for protection against oxidation is disclosed in U.S. Pat. No. 5,972,355.

The Biovert® system is a natural preservative system. Glucose is a natural source of energy. Glucose oxidase and lactoperoxidase are naturally occurring enzymes. Hypoiodite and hypothiocyanate are naturally occurring inorganic salts. However, hypothiocyanate is not permitted in organic products. Thus the Biovert® system is all natural, yet unsuitable for use in products certified organic by NOP or other certification agents.

The web site of arch Personal Care Products discloses Biomimetic Bodywash BP-6 (http://www.archchemicals.com/Fed/PC/Docs/BP-6%20Biomimetic%20Bodywash.pdf). This Bodywash comprises the Biovert system, pink grapefruit fragrance (essential oil) and citric acid (a plant extract). However, for a number of reasons, this Bodywash is not certifiable as organic. First, the hypothiocyanate in Biovert is not organic. Also, the formula contains other non-certifiable ingredients that total more than 39% of the formula. Thus, this reference and the prior art in general, fail to disclose or suggest an aqueous topical composition that satisfies one or more widely recognized organic certification standards and which comprises an antimicrobial-effective, natural preservative system and which has no synthetic preservatives. Thus, there remains a need for such a composition.

### OBJECTS OF THE INVENTION

A main object of the invention is to provide an aqueous topical composition that satisfies one or more widely recognized organic certification standards and which comprises an anti-microbial effective, natural preservative system having no synthetic preservatives.

Another object of the invention is to provide an aqueous topical composition that satisfies NOP and Ecocert certification standards and which comprises an anti-microbial effective, natural preservative system having no synthetic preservatives.

Another object of the invention is to provide an aqueous topical composition that satisfies NOP and Ecocert certification standards, wherein the preservative system efficacy meets a 5 log reduction standard commonly used in the field of cosmetics.

Another object of the invention is to provide an aqueous topical composition that satisfies NOP and Ecocert certification standards, wherein the preservative system comprises an in situ hypoiodite generator, at least one water based plant extract and at least one essential oil.

### SUMMARY

The present invention is an aqueous topical composition that satisfies one or more widely recognized organic certification standard. The composition comprises a preservative system comprising an in situ hypoiodite generator, an essential oil blend and a plant extract blend. Preferably, the composition contains no synthetic preservatives, especially parabens. If alcohol is present, preferably it is at no more than about 5% concentration. Suitable aqueous compositions may contain an oil phase, such as oil-in-water emulsions. Preferably, the composition satisfies a seven day, 5 log reduction standard for anti-microbial activity against molds (such as, Aspergillus niger), yeasts (such as, Candida albicans), gram positive bacteria (such as Staphylococcus aureus), gram negative bacteria (such as Pseudomonas aeruginosa), and enteric bacteria (such as E-coli).

### DETAILED DESCRIPTION

One of the dual pathways of the enzyme-based Biovert® system leads to the generation of hypothiocyanate ions. Hypothiocyanate ions are unacceptable in topical compositions certified organic. In the compositions according to the present invention, this pathway, along with the microbicidal activity that it contributes, is removed from the system. According to U.S. Pat. No. 5,607,681 (see column 13),
“Omission of thiocyanate resulted in failure against mould . . . . These results indicate that at least four components, namely glucose oxidase, glucose, iodide and thiocyanate, are essential components required to give broad spectrum anti-microbial activity.”

Thus, by removing the thiocyanate ions from the enzyme-based preservative system, the present invention goes against the prior art. Surprisingly, this modified Biovert® system is still useful for preservation, and when used in combination with certain natural ingredients, provides adequate preservation in aqueous products that are certifiable organic. This includes NOP and Ecocert certification, as well as others.

Throughout the specification, the term “hypoiodite generator”, specifically means that no hypothiocyanate ions are produced. The enzyme-based hypoiodite generator of the present invention consists of an oxidase, a substrate for the oxidase, a peroxidase and an iodide species, with no thiocyanate species. Surprisingly, without the hypothiocyanate pathway, the enzyme-based, organic hypoiodite generator is still useful as part of an anti-microbially-effective natural preservative system for certifiable organic preparations.

By “anti-microbially effective” or the like, we mean that a preserving system or preserving agent meets at least a 3 log reduction standard for molds, yeasts, gram positive and gram negative bacteria, and enteric bacteria, preferably a 5 log reduction standard. A “well preserved” composition is one that comprises an anti-microbially effective preservative system.

### Oxidase and Substrate

An appropriate oxidase is one which is able to catalyze the production of H₂O₂ by oxidizing a substrate in the presence of water and oxygen. Examples of useful oxidases include glucose oxidase or galactose oxidase. Appropriate substrates for these enzymes are D-glucose or galactose, respectively. Precursors of these compounds are also useful, for example oligomers or polymers that can break down into the smaller sugar units. The amount of oxidase used is preferably about 150-4,000 U/kg of the total composition, a unit (U) being defined herein as the amount of enzyme required to catalyze 1.0 μmole of substrate per minute at 25° C., under optimal conditions. More preferably the amount of oxidase is at about 200-3,000 U/kg, and most preferably about 300-2,5000 U/kg. Nevertheless, U.S. Pat. No. 5,607,681 teaches that the amount of glucose oxidase may be decreased to about 25 to 4000 U/kg, preferably 75 to 3000 U/kg glucose oxidase, if the composition further comprises “at least one antioxidant, for example 1 to 10000 mg/kg, preferably 50 to 5000 mg/kg butylated hydroxytoluene, α-tocopherol or esters thereof or ascorbic acid, esters or salts thereof”.

The substrate for the oxidase is preferably provided in an amount of at least about 0.5-50 g/kg of total composition, preferably at least 1 g/kg, and more preferably at least 2 g/kg.

### Peroxidase and Iodide Species

Compositions of the present invention include a source of iodide anions (I⁻). The anions are generally incorporated into the system in the form of salts, such as potassium and sodium iodide salts, or mixtures thereof. A preferred weight concentration of iodide ions is about 5-200 mg/kg of the total composition and preferably about 10-150 mg/kg of the total composition.

An appropriate peroxidase is one which, in the presence of hydrogen peroxide (H₂O₂), is capable of oxidizing the iodide ion into hypoiodite (OI⁻). The peroxidase may be, for example, lactoperoxidase, myeloperoxidase, or horseradish peroxidase. The amount of peroxidase is preferably at least about 10 to 100,000 U/kg, more preferably 100-25,000 U/kg and most preferably 250-10,000 U/kg, particularly 500-7000 U/kg.

Adhering to the concentrations discussed above, the oxidase, substrate, peroxidase and iodide ions may be added to the topical composition separately or in pre-mixed forms. For example, the iodide ions may be prepared as part of the substrate. For example, the substrate may be an aqueous solution of glucose or galactose mixed with one or more salts of iodide. When introduced into the substrate solution, the iodide ions are released from the salts. Subsequently, when the substrate solution is incorporated into the aqueous cosmetic preparation, the preparation becomes infused with iodide ions. Furthermore, the oxidase and peroxidase may be premixed in solution and added simultaneously to the aqueous preparation.

Advantageously, we are able to achieve a well preserved aqueous composition having no synthetic preservatives, including alcohol, that satisfies one or more widely recognized organic certification standards, including NOP and Ecocert (assuming the remainder of the composition is certifiable as organic). Such compositions are new and non-obvious, as evidenced by the lack of such compositions on the market, even though demand for organic certified products is high.

Furthermore, organically certified alcohol is permitted in certified organic products. Therefore, the option of including organic alcohol is also within the scope of this invention. Organic alcohol may be used for its preservation activity or for any of its other qualities. The concentration of organic alcohol will be dictated by its function, and in general, the concentration may be well below 15-20%. For example, in compositions of the present invention disclosed herein, no more than 5% organic alcohol is used to dissolve a blend of essential oils, the preservative activity of the organic alcohol being incidental. Alcohol at a 5% concentration is incapable of providing complete preservation of an aqueous cosmetic composition. Thus, the organic alcohol-containing compositions disclosed herein are distinguished from prior art compositions having alcohol at levels of 15-20%, or more.

The phrase “satisfies one or more widely recognized organic certification standards” is used in this specification. The term “satisfies”, by itself, only implies that a composition meets one of the various levels of organic labeling described above. One factor that makes a composition according to the present invention unique is that the composition satisfies one or more widely recognized organic certification standards and is well preserved while containing no synthetic preservatives or, in some cases, at most 5% organic alcohol. By “widely recognized”, we mean those standards promulgated by an agency that a person of ordinary skill in the art would recognize as authoritative and in force. At a minimum, this includes all government and government-sponsored certification programs, like NOP. Quasi governmental and non-governmental agencies, like Ecocert, are also included, when those agencies would be recognized as authoritative by a person of ordinary skill in the art.

A second component of the present invention is one or more water soluble plant extracts. Typically, plant extracts are suitable for use in certified organic products and may provide additional preservative activity. This may be desirable if the activity of the enzyme-based, organic hypoiodite generator alone, does not perform up to established standards. In that case, additional efficacy (either intensity or spectrum) will be required and various water soluble plants extracts have been identified for their antimicrobial activity. Of particular importance in the present invention are water soluble plant extracts that provide anti-mold activity. These include, for example, quillaja saponaria wood, olea europaea (olive leaf), citrus aurantium amara (bitter orange) flower, citrus medica limonum (lemon), citrus paradise (grapefruit) seed, salix alba (willow) bark, Sasa Kurilensis water (bamboo), Kappaphycus alvareziil (seaweed), Arachis hypogaea seed (oat), punica granatum (pomegranate) juice, vaccinium myrtillus (blueberry) leaf, fragaria vesca (strawberry) fruit and robus idaeus (raspberry) fruit extracts. Compositions of the present invention that comprise an oil phase may preferably include one or more oil soluble plant extracts to aid in the preservation of the oil phase. Useful oil soluble plant extracts include rosmarinas officinalis (rosemary) leaf, solanum lycopersicum (tomato) seed, hinokitiol and tocopherol. At present Tocobiol® (from BTSA, Biotecnologías Aplicadas, S.L.) is the only tocopherol acceptable under some (i.e. NOP) certification standards.

In general, other than the usual concerns in cosmetic and organic formulation, there are no restrictions on the concentrations of plant extracts used in formulations according to the present invention. Nevertheless, suitably preserved compositions have been readily achieved wherein the total concentration of plant extracts contributing to preservation is about 5% or less. Thus, well preserved organic compositions are possible, with relatively low levels of plant extracts.

An optional component of the present invention is one or more essential oils. Typically, essential oils are suitable for use in certified organic products and may provide additional preservative activity, including anti-oxidant activity. Compositions of the present invention that comprise an oil phase will preferably include one or more essential oils to aid in the preservation of the oil phase. Various essential oils have been identified for their antimicrobial activity. These include, for example, eugenia caryophyllus (clove) bud, lavendula angustifolia (lavender), cananga odorata (ylang ylang) flower, citrus grandis (grapefruit) peel, citrus medica limonum (lemon) peel and thymus vulgaris (thyme). Also useful are: origanum, sweet orange, lemongrass, Chinese cinnamon, rose, eucalyptus, peppermint, rose geranium, meadowsweet, Chinese anise, orris, cinnamon, rosemary, cumin, neroli, birch, Melissa balm, juniper, sweet fennel garlic, cajeput, sassafras, heliotrope, anise, mustard, fir, pine, parsley and violet. There are many other antimicrobially active essential oils, and as long as a supplier of organically certified material can be found, these may be used in the certified organic compositions of the present invention. A person skill in the art may construct a blend of essential oils that provides some necessary or backup preservative activity. Three blends that have been useful at about 0.5% of the total composition are as follows.

Blend 1: eugenia caryophyllus (clove) bud oil, lavendula angustifolia (lavender) oil, cananga odorata (ylang ylang) flower oil, citrus grandis (grapefruit) peel oil, citrus medica limonum (lemon) peel oil, and thymus vulgaris (thyme) oil.

Blend 2: citrus medica limonum (lemon) peel oil, eugenia caryphyllos (clove) flower oil and cinnamomum cassia (cinnamon) leaf oil.

Blend 3: pimenta officinalis (allspice) leaf oil and eugenia caryphyllos (clove) flower oil.

In general, other than the usual concerns in cosmetic and organic formulation, there are no restrictions on the concentrations of essential oils used in formulations according to the present invention. Nevertheless, suitably preserved compositions have been readily achieved wherein the total concentration of essential oils contributing to preservation is about 0.5% or less. When using essential oils, the aroma contributed to the product by the essential oils must be considered. If it is undesirable to have the preservative essential oils contribute to the overall aroma of the product, then total essential oil concentrations may be limited to whatever level is considered undetectable. The exact level may be determined by a person of ordinary skill in the art, however, concentrations of essential oil blends disclosed herein, up to about 0.5%, have contributed to the overall preservation of the product, while avoiding a substantial effect on a product's aroma, as detected by the human nose. Thus, well preserved organic compositions are possible, with relatively low levels of essential oils. Of course, if the aroma is considered a benefit for the product, then more essential oil may be used in the product.

The aqueous organic compositions of the present invention can be in any form, particularly water-in-oil emulsions. The aqueous phase of the composition may be any cosmetically acceptable water based material, such as deionized water, or a floral water. Because the preservative system of the present invention is particularly suited for water based certified-organic compositions, water may be present in various amounts, for example up to 30%, more preferably up to 60% and most preferably up to 80% of the composition, by weight.

The oil phase may be any cosmetically or pharmaceutically acceptable organic material which is substantially insoluble in water. For example, organic sunflower oil, almond oil, castor oil, coconut oil, palm oil, olive oil, pumpkin seed oil, safflower oil, or sesame seed oil.

The aqueous organic compositions of the present invention may comprise optional components, depending on the intended end use. In accordance with present invention, a material may only be included up to the concentration permitted by one or more widely recognized, valid, organic certification standards. By referencing any particular organic standard, a person of skill in the art may readily ascertain which materials are disallowed in organic products.

With that restriction, compositions of the present invention may, in general, contain ingredients having any of the functions listed in the International Cosmetic Ingredient Dictionary and Handbook, eleventh edition (2006) herein incorporated by reference, in its entirety. Examples of ingredients that provide one or more benefits to the skin or hair are abrasives, analgesics, antiageing agents, antidandruff agents, anti-inflammatory agents, antioxidants, antiperspirants, astringents, colorants; conditioners, cooling agents, deodorants, depilating agents, dyes, emollients, exfoliants, flavors, fragrances, hair fixatives, hair growth promoters, heating agents, humectants, lip plumping agents, lipids, magnetic agents, occlusives, optically active materials, pigments, protease inhibitors, self-tanners, structured water, sunscreens, thermochromic agents, waterproofing agents, whiteners and vitamins. Examples of ingredients that provide one or more benefits to the composition itself are absorbents, anticaking agents, antifoaming, antistatic agents, binders, buffers, bulking agents, chelating agents, cleansers, corrosion inhibitors, encapsulating agents, emulsion stabilizers, fibers, film-formers, foaming agents, gellants, opacifiers, oxidizing agents, pH adjusters, plasticizers, polymers, propellants, reducing agents, solvents, surfactants, suspending agents, UV absorbers and viscosity controlling agents.

Furthermore, compositions of the present invention may fall into any product category listed in the International Cosmetic Ingredient Dictionary and Handbook (eleventh edition). Examples of these include aftershave lotions, baby products, basecoats, bath products, beard softeners, blushers, body paints, cleansers, colognes, cuticle softeners, dentifrices, deodorants, depilatories, douches, eyeliners, eye lotions, eye makeups, face and neck preparations, foot powders, foundations, hair bleachers and colorants, hair conditioners, hair rinses, hair curlers, hair straighteners, lip aides, lip sticks, makeup bases, makeup pencils, manicuring preparations, mascara, masks, moisturizers, mouthwashes, mud packs, nail preparations, perfumes, powders, rouges, sachets, shampoos, shaving aides, self tanners, suntan preparations, talcum and tonics.

The preservation of an all-natural, certifiable-organic cosmetic preparation as disclosed herein, is typically comparable to or superior to conventional cosmetic preservation. That a preservative system of all natural ingredients, in a certifiable organic composition, could perform as well or better than conventional chemical preservatives, is surprising. The invention is further illustrated by the following non-limiting examples, which include compositions that readily meet a 5 log reduction standard within seven days, for molds, yeasts, gram positive, gram negative and enteric bacteria.

### EXAMPLE 1

### Organic Moisture Lotion

The oil-in-water compositions 2 and 3 of table 1 were formed by adding a hypoiodite generator at 38° C., after the emulsions were formed. Compositions 2 and 3 are anti-microbial-effective, while composition 1, without a hypoiodite generator, failed micro challenge testing for mold, yeast, Staphylococcus aureus, Pseudomonas aeruginosa, and enteric bacteria. Thus, composition 1 may be certifiable organic, but the combination of aqueous plant extract (rosemary), tocopherol and citric acid are not antimicrobial-effective. In contrast, compositions 2 and 3 are well preserved, meaning the preservative system is antimicrobial-effective. This is unexpected, considering all of the antimicrobial activity lost with the removal of thiocyanate ions from the Biovert substrate and the fact that there is no alcohol in these compositions. Simultaneously, the removal of the thiocyanate ions renders compositions 2 and 3 certifiable organic (as far as the ingredients are concerned).

TABLE 1

| | 1 (%) | 2 (%) | 3 (%) |
|---|---|---|---|
| Oil Phase | | | |
| organic sesame | 20.00 | 20.00 | 20.00 |
| seed oil | | | |
| organic soy lecithin | 3.00 | 3.00 | 3.00 |
| tocopherol | 1.00 | 1.00 | 1.00 |
| Water Phase | | | |
| deionized water | 66.30 | 62.25 | 65.25 |
| corn starch | 2.50 | 2.50 | 2.50 |
| citric acid | 0.10 | 0.10 | 0.10 |
| rosemary extract | 0.10 | 0.10 | 0.10 |
| glycerine | 7.00 | 7.00 | 7.00 |
| water/glucose/potassium | 0 | 1.00 | 1.00 |
| iodide | | | |
| water/glucose | 0 | 0.05 | 0.05 |
| oxidase/lactoperoxidase | | | |

### EXAMPLE 2

### Hydrating Body Lotion

In table 2, compositions 4 and 5, without a hypoiodite generator, failed micro challenge testing for mold, yeast, Staphylococcus aureus, Pseudomonas aeruginosa, and enteric bacteria. Thus, in composition 4, the combination of tocopherol, alcohol and citric acid does not provide effective antimicrobial activity. Furthermore, the addition of an antimicrobial essential oil blend, in composition 5, still does not provide effective antimicrobial activity. In contrast, composition 6 is anti-microbial-effective. The composition meets a seven day, 5 log reduction standard, remains microbially clean to at least 3 weeks and when reinoculated, again satisfied a seven day 5 log reduction. By selecting certified organic versions of the ingredients (where possible), composition 6 may be certifiable organic. That composition 6 is antimicrobially effective is unexpected, considering all of the antimicrobial activity lost with the removal of thiocyanate ions from the Biovert substrate. Simultaneously, the removal of the thiocyanate ions renders composition 6 certifiable organic (as far as the ingredients are concerned). Thus, composition 6 represents a well preserved, aqueous composition that is certifiable organic by one or more widely recognized organic certification standards.

TABLE 2

| Sequence | | 4 (%) | 5 (%) | 6 (%) |
|---|---|---|---|---|
| 1 | deionized water | 38.40 | 37.90 | 37.98 |
| 1 | waxy maize | 2.50 | 2.50 | 2.50 |
| 2 | glycerine | 7.00 | 7.00 | 7.00 |
| 3 | deionized water | 9.80 | 9.80 | 9.80 |
| 3 | xanthan gum | 0.20 | 0.20 | 0.20 |
| 4 | deionized water | 9.50 | 9.50 | 9.50 |
| 4 | veegum pure | 0.50 | 0.50 | 0.50 |
| 5 | sunflower seed oil | 20.00 | 20.00 | 9.00 |
| 5 | coconut oil | | | 6.00 |
| 5 | organic soy lecithin | 3.00 | 3.00 | 2.50 |
| 5 | tocopherol | 1.00 | 1.00 | 1.00 |
| 5 | beeswax | | | 2.00 |
| 5 | palm fruit oil | | | 2.00 |
| 6 | silica beads | 2.00 | 2.00 | 3.00 |
| 7 | deionized water | 1.00 | 1.00 | 1.00 |
| 7 | citric acid | 0.10 | 0.10 | 0.10 |
| 8 | alcohol denatured | 5.00 | 5.00 | 5.00 |
| 8 | essential oil blend | | 0.50 | 0.50 |
| 9 | water/glucose/potassium | | | 0.40 |
| | iodide | | | |
| 9 | water/glucose | | | 0.02 |
| | oxidase/lactoperoxidase | | | |

### EXAMPLE 3

### Nourishing Face Lotion

The compositions of table 3 are additional examples of an aqueous topical compositions that are certifiable organic by one or more widely recognized organic certification standards and which comprises an antimicrobial-effective preservative system. In the case of compositions 7 and 8, there are no artificial preservatives, alcohol not being used in those compositions. The composition meets a seven day, 5 log reduction standard, remains microbially clean to at least 3 weeks and when reinoculated, again satisfied a seven day 5 log reduction.

TABLE 3

| | | 7(%) | 8(%) | 9(%) |
|---|---|---|---|---|
| 1 | deionized water | 57.83 | 60.365 | 63.04 |
| 1 | corn starch | 3.00 | 3.000 | 2.50 |
| 2 | xanthan gum | | | 0.20 |
| 2 | veegum pure | | | 0.50 |
| 2 | sunflower oil | 14.10 | 14.100 | 11.00 |
| 2 | palm oil | 2.00 | 9.375 | 2.00 |
| 2 | coconut oil | 2.00 | | 4.00 |
| 2 | shea butter | 3.00 | | 1.00 |
| 2 | cocoa butter | 3.00 | | 1.00 |
| 2 | soy lecithin | 3.00 | 3.00 | 2.50 |
| 2 | tocopherol | 1.00 | 1.00 | 0.50 |
| 2 | rosemary leaf oil | 0.01 | 0.01 | 0.01 |
| 2 | glycerine | 7.00 | 7.00 | 7.00 |
| 3 | olive leaf ext | | 0.50 | |
| 3 | quillaja saponaria extract | 2.00 | 0.10 | 0.10 |
| 3 | alcohol denatured | | | 3.00 |
| 3 | essential oil blend | 0.50 | 0.45 | 0.50 |
| 4 | citric acid | 0.01 | | 0.10 |
| 5 | deionized water | 0.50 | | |
| 5 | patchouli oil | | 0.025 | |
| 5 | rose oil | | 0.025 | |
| 6 | water/glucose/potassium | 1.00 | 1.00 | 1.00 |
| | iodide | | | |
| 7 | water/glucose | 0.05 | 0.05 | 0.05 |
| | oxidase/lactoperoxidase | | | |

What is claimed is:

1. A well preserved aqueous composition that satisfies one or more widely recognized organic certification standards, comprising an antimicrobial-effective, natural preservative system, the preservative system comprising:
an in situ hypoiodite generator; and
no synthetic preservatives.

2. A composition according to claim 1 wherein the organic certification standards are chosen from the National Organic Program, Ecocert, the Organic Japanese Agricultural Standard, National Programme for Organic Production, the Soil Association Standards for Health and Beautycare Products, and EU Organic Regulation EC2092/91

3. A composition according to claim 2, wherein the preservative system meets a 5 log reduction standard for all of molds, yeasts, gram positive bacteria, gram negative bacteria and enteric bacteria.

4. A composition according to claim 2, wherein the preservative system further comprises at least one water soluble plant extract that has anti-fungal properties.

5. A composition according to claim 4 comprising one or more plant extracts chosen from quillaja saponaria wood, olea europaea (olive leaf), citrus aurantium amara (bitter orange) flower, citrus medica limonum (lemon), citrus paradise (grapefruit) seed, salix alba (willow) bark, Sasa Kurilensis water (bamboo), Kappaphycus alvareziil (seaweed), Arachis hypogaea seed (oat), punica granatum (pomegranate) juice, vaccinium myrtillus (blueberry) leaf, fragaria vesca (strawberry) fruit and robus idaeus (raspberry) fruit extracts.

6. A composition according to claim 2 further comprising an oil phase, the oil phase comprising one or more of organic sunflower oil, organic almond oil, organic castor oil, organic coconut oil, organic palm oil, organic olive oil, organic pumpkin seed oil, organic safflower oil, or organic sesame seed oil.

7. A composition according to claim 6, wherein the preservative system further comprises at least one essential oil that has anti-microbial properties.

8. A composition according to claim 7 comprising one or more essential oils chosen from eugenia caryophyllus (clove) bud, lavendula angustifolia (lavender), cananga odorata (ylang ylang) flower, citrus grandis (grapefruit) peel, citrus medica limonum (lemon) peel, thymus vulgaris (thyme), cinnamomum cassia (cinnamon) leaf oil, and pimenta officinalis (allspice) leaf oil.

9. A composition according to claim 2 wherein:
iodide anions are present at a concentration of at least about 5 mg/kg based on total weight of the composition,
glucose is present at a concentration of at least about 0.2 g/kg based on total weight of the composition,
glucose oxidase is present at at least about 150 U/kg; and
peroxidase is present at at least about 10 U/kg.

10. The topical composition of claim 1 that comprises at least about 30% water.

11. The topical composition of claim 1 that comprises at least about 60% water.

12. The topical composition of claim 1 that comprises at least about 80% water.

13. A well preserved aqueous composition that satisfies one or more widely recognized organic certification standards, comprising an antimicrobial-effective, preservative system, the preservative system comprising:
an in situ hypoiodite generator; and
organic alcohol up to about 5%.
